(19) United States
(12) Patent Application Publication
(10) Pub. No.: US 2001/0020090 A1
(43) Pub. Date: Sep. 6, 2001

(54) Process for the acid-catalyzed, hydrolytic depolymerization of cellulose ethers

(75) Inventors: Winfried Becker, Eppstein (DE); Alf Hammes, Mainz-Kastel (DE)

Correspondence Address: Clariant Corporation, Industrial Property Department, 4331 Chesapeake Drive, Charlotte, NC, 28216, US

(73) Assignee: Clariant GmbH

(21) Appl. No.: 09/798,861

(22) Filed: Mar. 1, 2001

(30) Foreign Application Priority Data
Mar. 1, 2000 (DE) 100 09 642.5

(51) Int. Cl. C08B 11/20; C07H 3/00

(52) U.S. Cl. 536/88; 536/84; 536/89; 536/124

(57) ABSTRACT

In the process according to the invention, the cellulose ether to be depolymerized is depolymerized in the form of an acid slurry in an apparatus which consists of an acid-resistant material and is provided with an inlet and outlet, where the inlet and outlet are separated from one another by a filter element, at a temperature above the flocculation temperature of the depolymerized cellulose ether in the space between the inlet and the filter element, subsequently neutralized and then separated from the filtrate by releasing the filtrate, which, in contrast to the cellulose ether, is not retained by the filter element. If necessary, this can be followed by purification and drying of the cellulose ether.

### FIELD OF THE INVENTION

The invention relates to a process for the acid-catalyzed, hydrolytic depolymerization of cellulose ethers and subsequent separation and, if necessary, purification and drying of the depolymerized cellulose ether without changing the apparatus.

### BACKGROUND OF THE INVENTION

The depolymerization of cellulose ethers for the specific setting of the desired solution viscosity of the resultant product in aqueous solution has been known for some time and can be achieved in many ways. In particular, degradation to give extremely low-viscosity products has attracted considerable attention since these products can advantageously be employed, inter alia, as coating material for pharmaceutical active ingredients or seed, but also, for example, as protective colloid in suspension polymerization. The term extremely low-viscosity products is applied below to cellulose ethers whose Hoppler viscosity, measured in 2.0% (absolutely dry) solution in water at 20° C., is not greater than 50 mPas.

The processes employed for the degradation of cellulose ethers, besides acidcatalyzed, hydrolytic cleavage of the acetal bond, include, inter alia, oxidative degradation and degradation by high-energy radiation or microorganisms/enzymes.

Simple hydrolytic degradation processes using inorganic or organic acids are described, for example, in US-A-1,679,943, US-A-1,943,461, EP-B-0 497 985 and EP-A-0 210 917.

Hydrolytic degradation is functional-group-neutral and gentle and can be employed for the production of extremely low-viscosity products. However, if the cellulose ether to be degraded is in relatively large dilution in the aqueous medium, losses of yield are virtually unavoidable.

The lower the mean degree of polymerization of the product to be prepared, the greater the risk of dissolution or partial dissolution and sticking of the material in aqueous suspension. For this reason, common processes by means of which cellulose ethers in aqueous suspension can be separated from the aqueous phase, such as, for example, decanters or filter presses, can only be employed to a limited extent, in particular in the case of extremely low-viscosity products.

### SUMMARY OF THE INVENTION

The object of the present invention was therefore to develop a process for the depolymerization of cellulose ethers by acid-catalyzed, hydrolytic degradation and subsequent separation and, if necessary, purification and drying of the depolymerized cellulose ether which can be carried out without changing the apparatus.

### DESCRIPTION OF THE DRAWING

FIG. 1 is a partial longitudinal sectional view of an enameled pressure filter in accordance with the present invention.

### DESCRIPTION OF THE PREFERRED EMBODIMENTS

The invention relates to a process for the acid-catalyzed, hydrolytic depolymerization of cellulose ethers with subsequent separation and, if necessary, purification and drying of the depolymerized cellulose ethers without changing the space used, which comprises, in a sealed, pressure-tight, heatable space consisting of acid-resistant materials, provided with an inlet and an outlet which are separated from one another by a filter element, and fitted with a stirrer, depolymerizing the cellulose ether to be depolymerized, which is introduced through the inlet and retained by the filter element, in the form of an acidic slurry with stirring at a temperature above the flocculation temperature of the depolymerized cellulose ether, if necessary under pressure, and, when the depolymerization is complete, neutralizing the cellulose ether by introducing caustic lye and, if desired, additives, and subsequently, by opening the outlet, under the effect of gravity and/or if desired by applying an external pressure to the space, separating off the cellulose ether from the filtrate, which is not retained by the filter element, and, if necessary, subsequently purifying the cellulose ether by introducing water or a mixture of water and one or more organic suspension media one or more times at a temperature above the flocculation temperature of the depolymerized cellulose ether, and subsequently releasing the water or water/suspension medium mixture, and subsequently, if necessary, drying the cellulose ether by applying a vacuum to the space or passing a gas through the space.

The term ‘space’ here is taken to mean, for example, a tank, a vessel, a reactor or an apparatus.

In principle, it is also possible to use embodiments of the space in which the space is provided with one or more inlets and outlets, where the inlets and outlets are in each case separated from one another by one or more filter elements.

A particularly surprising feature of the process is that the filter element does not become blocked in spite of the in some cases considerable tack of the partially swollen cellulose ether, and effective separation of the depolymerized, flocculated product from the aqueous reaction slurry is possible.

In the acidic slurry, the weight ratio between the liquid medium and the cellulose ether to be depolymerized is preferably at least 2:1, particularly preferably in the range from 4:1 to 10:1.

The temperature of the slurry is preferably at least 50° C., particularly preferably at least 80° C.

The slurries employed are preferably aqueous slurries, i.e. the liquid medium in the slurries is water.

Besides water, it is also possible to use mixtures of water and one or more organic suspension media as liquid media for the slurries. Preferred organic suspension media are acetone, t-butanol and ethers, such as, for example, diethyl ether or higher homologues, and dimethoxyethane and cyclic ethers.

The acidic slurry is preferably prepared by initially introducing the water or the water/suspension medium mixture at a temperature above the flocculation temperature of the cellulose ether to be depolymerized, then introducing the cellulose ether to be depolymerized through the inlet into the space, and subsequently introducing acid, where the amount of liquid used should be such that good mixing by means of stirring is possible.

The pH of the acidic slurry is preferably in the range from 0.1 to 5, particularly preferably in the range from 1.5 to 4 and in particular in the range from 2 to 3.5.

Suitable for setting the acidic pH of the slurry are inorganic and/or organic acids. Preferred acids are in particular hydrochloric acid, sulfuric acid, nitric acid and phosphoric acid, and mixtures thereof in any desired appropriate concentration.

Depending on the desired degree of polymerization of the depolymerized cellulose ether, the pH of the acidic slurry and the depolymerization time are matched to one another.

The depolymerization time is preferably from 1 minute to 10 hours, particularly preferably from 10 minutes to 2 hours and in particular from 10 to 60 minutes.

The Hoppler viscosity of the depolymerized cellulose ether, measured in 2.0% (absolutely dry) solution in water at 20° C., is preferably not greater than 50 mPas, particularly preferably not greater than 20 mPas.

During neutralization of the slurry, its pH is preferably adjusted to a value of from 6.5 to 9, particularly preferably from 7 to 8, using caustic lye.

The neutralization is preferably carried out using an aqueous solution of sodium hydroxide. The concentration of the caustic lye is preferably in the range from 10 to 35%.

The residual moisture content of the pressed-out, depolymerized cellulose ether can be up to 90%, depending on the degree of etherification, but is generally less than 60%.

The separation of the filtrate from the depolymerized cellulose ether is advantageously followed by purification of the cellulose ether, during which water or a water/suspension medium mixture is applied to the cellulose ether one or more times and subsequently released. Preferred organic suspension media are acetone, t-butanol and ethers, such as, for example, diethyl ether or higher homologues, and dimethoxyethane and cyclic ethers.

Also advantageous is subsequent drying of the cellulose ether by application of a vacuum to the space or passing an inert gas through the space. Preferred gases for the latter operation are nitrogen, air or superheated steam.

The cellulose ethers to be depolymerized are preferably carboxymethylcellulose, carboxymethylhyd roxypropylcellu lose, methylcellulose, methylhyd roxyethylcellulose and methylhyd roxypropylcellulose.

The filter element is preferably a device which is impermeable to particles whose diameter is greater than 2 μm. Particular preference is given to filter elements which are impermeable to particles having a diameter of greater than 5 μm.

Preferred stirrers are those which have adjustable height and can be heated if desired. The geometry of the stirrer is unimportant so long as effective mixing of the slurry is ensured and the stirrer is, where necessary, capable of discharging the depolymerized cellulose ether from the space.

If desired, the space additionally has a compressed-air inlet, a compressed-air outlet, a gas inlet, a gas outlet, a vacuum connection, a solids discharge for the depolymerized cellulose ether and/or one or more metering devices.

The acid-resistant materials which are in contact with the acidic slurry are preferably enamel or acid-resistant metal alloys, such as, for example, Hastelloy C, tantalum and/or acid-resistant plastics, such as, for example, polypropylene.

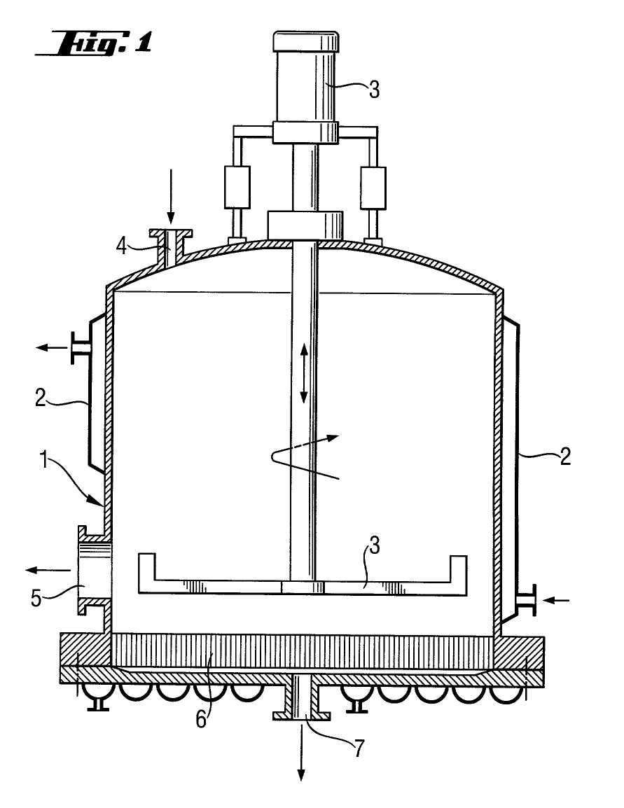

The process is particularly preferably carried out using an enameled pressure filter (1), as shown diagrammatically in FIG. 1, or a comparable apparatus, which is provided with an inlet (4), an outlet (7), a heating jacket (2), a height-adjustable stirrer (3), a solids discharge (5) for the depolymerized cellulose ether and a filter base (6). All non-enameled parts which are in contact with the acidic slurry consist of an acid-resistant material.

The present invention thus also relates to the use of an enameled pressure filter (1) which is provided with an inlet (4), an outlet (7), a heating jacket (2), a stirrer (3), a solids discharge (5) and a filter base (6) in which all non-enameled parts consist of an acid-resistant material, for the depolymerization of cellulose ethers.

Claims:

1. A process for the acid-catalyzed, hydrolytic depolymerization of cellulose ethers with subsequent separation and, if necessary, purification and drying of the depolymerized cellulose ethers without changing the space used, which comprises, in a sealed, pressure-tight, heatable space consisting of acid-resistant materials; provided with an inlet and an outlet which are separated from one another by a filter element, and fitted with a stirrer, depolymerizing the cellulose ether to be depolymerized, which is introduced through the inlet and retained by the filter element, in the form of an acidic slurry with stirring at a temperature above the flocculation temperature of the depolymerized cellulose ether, if necessary under pressure, and, when the depolymerization is complete, neutralizing the cellulose ether by introducing caustic lye and, if desired, additives, and subsequently, by opening the outlet, under the effect of gravity and/or if desired by applying an external pressure to the space, separating off the cellulose ether from the filtrate, which is not retained by the filter element, and, if necessary, subsequently purifying the cellulose ether by introducing water or a mixture of water and one or more organic suspension media one or more times at a temperature above the flocculation temperature of the depolymerized cellulose ether and subsequently releasing the water or water/suspension medium mixture, and subsequently, if necessary, drying the cellulose ether by applying a vacuum to the space or passing a gas through the space.

2. The process as claimed in claim 1, wherein, in the slurry, the weight ratio between the liquid medium and the cellulose ether to be depolymerized is at least 2:1.

3. The process as claimed in claim 1, wherein the temperature of the slurry is at least 50° C.

4. The process as claimed in claim 1, wherein the slurry is an aqueous slurry.

5. The process as claimed in claim 1, wherein the liquid medium of the slurry is a mixture of water and one or more organic suspension media.

6. The process as claimed in claim 1, wherein the acidic slurry is prepared by initially introducing water or a water/suspension medium mixture at a temperature above the flocculation temperature of the cellulose ether to be depolymerized, then introducing the cellulose ether to be depolymerized, and subsequently introducing an acid.

7. The process as claimed in claim 1, wherein the pH of the acidic slurry is from 0.1 to 5.

8. The process as claimed in claim 1, wherein the pH of the acidic slurry and/or the depolymerization time are matched to one another in accordance with the desired degree of polymerization of the depolymerized cellulose ether.

9. The process as claimed in claim 1, wherein the depolymerization time is from 1 minute to 10 hours.

10. The process as claimed in claim 1, wherein the Hoppler viscosity of the depolymerized cellulose ether, measured in 2.0% (absolutely dry) solution in water at 20° C., is not greater than 50 mPas.

11. The process as claimed in claim 1, wherein the pH of the neutralized slurry is from 6.5 to 9.

12. The process as claimed in claim 1, wherein the cellulose ether to be depolymerized is carboxymethylcellulose, carboxymethylhydroxypropylcellulose, methylcellulose, methylhydroxypropylcellulose or methylhydroxypropylcellulose.

13. The process as claimed in claim 1, wherein the filter element is impermeable to particles having a diameter greater than 2 μm.

14. The process as claimed in claim 1, wherein the space is additionally provided with a compressed-air inlet, a compressed-air outlet, a gas inlet, a gas outlet, a vacuum connection, a solids discharge and/or one or more metering devices.

15. The process as claimed in claim 1, wherein the acid-resistant materials are enamels, plastics, metals and/or metal alloys.

16. The process as claimed in claim 1, wherein the sealable space is an enameled pressure filter (1) which is provided with an inlet (4), an outlet (7), a heating jacket (2), a height-adjustable stirrer (3), a solids discharge for the depolymerized cellulose ether (5) and a filter base (6), in which all non-enameled parts in contact with the acidic slurry consist of an acid-resistant material.

17. A process for the depolymerization of cellulose ethers, which is conducted in an enameled pressure filter (1) provided with an inlet (4), an outlet (7), a heating jacket (2), a stirrer (3), a solids discharge (5) and a filter base (6), whereby all non-enameled parts consist of said filter (1) consist an acid-resistant material.
